(19) United States
(12) Patent Application Publication
(10) Pub. No.: US 2017/0172439 A1
(43) Pub. Date: Jun. 22, 2017

(54) ELECTRODES AND SENSORS HAVING NANOWIRES

(71) Applicant: North Carolina State University, Raleigh, NC (US)

(72) Inventors: YONG ZHU, Raleigh, NC (US); SHANSHAN YAO, Raleigh, NC (US); LINGNAN SONG, Raleigh, NC (US); AMANDA MYERS, Raleigh, NC (US)

(21) Appl. No.: 15/127,455

(22) PCT Filed: Apr. 7, 2015
(86) PCT No.: PCT/US15/24696

§ 371 Date: Sep. 20, 2016

Related U.S. Application Data
(60) Provisional application No. 61/976,086, filed on Apr. 7, 2014.

(51) Int. Cl. A61B 5/0408; B32B 15/18; B32B 15/02; B32B 15/14; B32B 15/04; A61B 5/053; B32B 37/18; B29C 70/18; B29C 70/88; A61B 5/0478; A61B 5/0492; B32B 38/10; B32B 37/10

(52) U.S. Cl. CPC A61B 5/0408; B32B 38/10; B32B 15/18; B32B 15/02; B32B 15/14; B32B 15/043; B32B 37/1018; B32B 37/18; B29C 70/18; B29C 70/885; A61B 5/0478; A61B 5/0492; A61B 5/0531; B32B 2307/202; B32B 2457/00; B32B 2535/00; B32B 2313/04; B32B 2311/08; B32B 2311/30; B32B 2305/07; B32B 2305/10; B32B 2262/105; B32B 2260/021; B32B 2260/046; B32B 2250/02; B29L 2031/3406

(57) ABSTRACT

Disclosed are various embodiments for electrodes and sensors having nanowires. According to an embodiment as described, a dry sensor is provided. Nanowires, such as silver nanowires, are positioned within a polymer material, such as polydimethylsiloxane (PDMS) to form an electrode. A conductive element is attached to the electrode during its formation. Example conductive elements include, but are not limited to, a contact or a wire that may be communicatively coupled to medical equipment.

### CROSS-REFERENCE TO RELATED APPLICATION(S)

This invention claims the benefit of and priority to co-pending U.S. Provisional Patent Application No. 61/976,086 entitled “ELECTRODES AND SENSORS HAVING NANOWIRES AND ASSOCIATED METHODS,” filed on Apr. 7, 2014, which is hereby incorporated by reference herein in its entirety.

### GOVERNMENT RIGHTS NOTICE

This invention was made with government support under grant number EEC-1160483, awarded by the National Science Foundation (NSF). The Government has certain rights in the invention.

### BACKGROUND

A rising interest in continuous personal health monitoring has drawn attention to the bioelectrodes currently in use, such as in electrocardiograms (ECG), electromyograms (EMG), and electroencephalograms (EEG), and the issues associated with them. The silver/silver chloride (Ag/AgCl) pre-gelled electrodes can be reliable and cost effective, however the required use of an electrolytic gel limits the long term use. For example, the gel dries out over time causing skin irritation and a degradation in signal quality. Dry electrodes, however, are limited by high skin-electrode impedance, poor signal quality, durability, and complex fabrication processes which can lead to a high cost of manufacturing.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1A is a schematic diagram showing an example fabrication process and drawing of an example AgNW/PDMS electrode having a snap according to various embodiments.

FIG. 1B is a schematic diagram showing an example fabrication process and drawing of an example AgNW/PDMS electrode having a lead wire according to various embodiments.

FIG. 2 shows a graph of line resistance of an example AgNW/PDMS electrode when strained to 80% according to various embodiments.

FIG. 3 shows a graph of skin-electrode impedance trend with varying application pressures according to various embodiments.

FIG. 4 shows a stationary ECG taken with an Ag/AgCl electrode and an example AgNW/PDMS electrode according to various embodiments.

FIG. 5A shows a fabrication process flow of an example capacitive multifunctional sensor according to various embodiments.

FIG. 5B shows a cross-sectional view of the example capacitive multifunctional sensor of FIG. 5A according to various embodiments.

FIG. 6 shows schematic diagrams depicting three sensing modalities of the example capacitive multifunctional sensor according to various embodiments.

FIG. 7A shows a relative capacitance change ΔC/C₀ versus tensile strain for stretching and releasing of a fabricated capacitive sensor according to various embodiments.

FIG. 7B shows a relative capacitance change ΔC/C₀ versus tensile strain for two measurements of a fabricated capacitive sensor, the second measurement being conducted after the sensor is stretched and released 100 cycles according to various embodiments.

FIG. 7C shows a one pixel sensor on a thumb joint according to various embodiments.

FIG. 7D shows a relative capacitance change and strain associated with thumb flexure when holding a thumb up, to making a fist, and back to a relaxed state according to various embodiments.

FIG. 7E shows a schematic diagram of the patellar reflex experiment according to various embodiments.

FIG. 7F shows a change in capacitance and strain caused by knee motion in patellar reflex according to various embodiments.

FIG. 7G shows a relative capacitance change and strain versus time for various human motions according to various embodiments.

FIG. 8A shows a relative capacitance change ΔC/C₀ of one pixel versus normal pressure for two consecutive measurements according to various embodiments.

FIG. 8B shows a response of a pressure sensor to water droplets according to various embodiments.

FIG. 8C shows a sensor array with a PDMS mold in the shape of “I” and the resulting map of capacitance change according to various embodiments.

FIG. 9A shows a change in capacitance for one pixel before and after repeated finger touching according to various embodiments.

FIG. 9B shows a change in capacitance for strong finger press (pressing mode) and gentle finger touch (proximity mode) according to various embodiments.

FIG. 9C shows a capacitance change as a function of the distance between sensor and finger when the finger pad approaches and leaves the sensor according to various embodiments.

FIG. 9D shows a capacitance change as a function of the distance between sensor and finger when the fingertip approaches and leaves the sensor according to various embodiments.

FIG. 10A shows a fabrication procedure for an AgNW/PDMS flexible patch antenna according to various embodiments.

FIG. 10B shows a schematic diagram for a microstrip patch antenna according to various embodiments.

FIG. 10C shows a schematic diagram for a 2-element patch array according to various embodiments.

FIG. 11A shows a simulated radiation pattern for a AgNW/PDMS microstrip patch antenna according to various embodiments.

FIG. 11B shows a simulated radiation pattern for a AgNW/PDMS 2-element patch array according to various embodiments.

FIG. 12 shows a comparison of a measured and a simulated reflection coefficient for the AgNW/PDMS microstrip patch antenna according to various embodiments.

FIG. 13 shows a comparison of a measured and a simulated reflection coefficient for the AgNW 2-element patch array according to various embodiments.

FIG. 14 shows a graph of an estimated and a simulated radiation efficiency for the microstrip patch antenna according to various embodiments.

FIG. 15A shows a measured and simulated normalized radiation pattern in E-plane according to various embodiments.

FIG. 15B shows a measured and simulated normalized radiation pattern in H-plane for the microstrip patch antenna at frequency of 2.92 GHz according to various embodiments.

FIG. 16A shows a measured frequency response of reflection coefficient for the AgNW/PDMS microstrip patch antenna under tensile strains from 0% to 15% according to various embodiments.

FIG. 16B shows a comparison of a simulated and a measured resonant frequency during stretch and release and measured resonant frequency during stretch and release for the AgNW/PDMS microstrip patch antenna under tensile strain from 0% to 15% according to various embodiments.

FIG. 17A shows photographs of a stretchable microstrip patch antenna composed of AgNW/PDMS flexible conductor in a relaxed state according to various embodiments.

FIG. 17B shows photographs of a stretchable microstrip patch antenna composed of AgNW/PDMS flexible conductor in a bent state according to various embodiments.

FIG. 17C shows photographs of a stretchable microstrip patch antenna composed of AgNW/PDMS flexible conductor in a twisted state according to various embodiments.

FIG. 17D shows photographs of a stretchable microstrip patch antenna composed of AgNW/PDMS flexible conductor in a rolled state according to various embodiments.

FIG. 18 shows a reflection coefficient versus frequency according to various embodiments.

FIG. 19 is a schematic diagram of an example RFID according to various embodiments.

FIG. 20A shows an example planar inductor based on AgNW/PDMS stretchable conductors according to various embodiments.

FIG. 20B shows a real impedance measured from the antenna as a function of frequency according to various embodiments.

FIG. 20C shows a Lorentzian fits of four representative impedance spectra of the LC sensor under various strains according to various embodiments.

FIG. 20D shows a resonant frequency of the LC sensor as a function of tensile strain applied on the planar inductor according to various embodiments.

FIG. 21 is a flowchart illustrating one example of a method employed to fabricate an electrode according to various embodiments.

FIG. 22 is a flowchart illustrated one example of a method employed to fabricate a stretchable patch antenna or a capacitive multifunctional sensor according to various embodiments.

### DETAILED DESCRIPTION

The present disclosure relates to electrodes and sensors having nanowires. With the recent progress of robotic systems, prosthetics and wearable medical devices, efforts have been devoted towards realization of highly sensitive and skin-mountable sensors. Sensors with various sensing capabilities could help the robotics and prosthetic devices mimic how a real-world object “feel” during interactions, obtain biosignals such as finger touching and body motions sent from human, and provide feedback information during actuating. Besides, wearable sensors that can be embedded into clothes or directly wrap around non-planar and biological surfaces are widely used to monitor human body motions and offer new opportunities for real-time health/wellness monitoring. For those applications, stretchability of the sensors are generally required in addition to flexibility. Wearable wireless communication is important to convey sensory data and provide remote diagnosis, and a radio frequency antenna is a critical component for the wireless communication.

Antennas are conventionally fabricated by printing or etching metal patterns on rigid substrates, which can easily crease and even fail to function properly when subjected to mechanical deformation (e.g., stretching, folding or twisting). Thus, development of flexible, stretchable, and conformal antennas calls for new electronic materials and/or new device configurations.

In accordance with embodiments as described herein, a multifunctional wearable sensor may be formed using highly conductive and stretchable silver nanowires (AgNWs) conductors, which enable the detection of strain (up to 50%), pressure (up to ˜1.2 MPa) and finger touch on a single platform. The sensors exhibit large stretchability, high sensitivity, fast response time (˜40 ms), and good pressure mapping function. Such sensors can be readily mounted onto a human body to monitor the skin strain associated with thumb flexing, knee jerk, and other human motions including walking, running, jumping, and squatting. In an example, the present subject matter may be applied as a bioelectronic electrode. For example, the electrode may be used in medical equipment for EMG, ECG, EEG, hydration sensing, muscle monitoring, and impedance-measurement. Additional details of these uses are provided herein.

In accordance with embodiments, disclosed herein is a class of microstrip patch antennas that are stretchable, mechanically tunable and reversibly deformable. The radiating element of the antenna consisted of a highly conductive and stretchable material with AgNWs embedded in the surface layer of an elastomeric substrate. More specifically, a 3-GHz microstrip patch antenna and a 6-GHz, 2-element patch array can be fabricated. Since a resonant frequency increases with increasing tensile strain, the antenna can be used for wireless strain sensing. Finally, the antennas maintain the same spectral properties under severe bending, twisting, and rolling.

Electrodes and sensors having nanowires and associated methods are disclosed herein. According to an aspect, a method of producing a sensor is provided. The method includes positioning nanowires, such as AgNWs, within a polymer material, such as polydimethylsiloxane (PDMS), to form an electrode. The method also includes attaching a conductive element to the electrode. Example conductive elements include, but are not limited to, a contact, a button, and a wire. In an example, a conductive element may be attached by use of a liquid metal. The metal in this example may be liquid at room temperature for use in reinforcing the contact. An example metal includes, but is not limited to, eutectic gallium indium (EGaIn).

According to another aspect, a method of producing a capacitive sensor is provided. The method includes positioning a first plurality of nanowires within a first polymer material. The method also includes positioning a second plurality of nanowires within a second polymer material. Further, the method includes attaching a non-conductive material to a side of the first polymer material and to a side of the second polymer material.

In accordance with embodiments, a dry silver nanowire-based electrode is employed for use in electrocardiogram (ECG or EKG) equipment, electroencephalogram (EEG) equipment, electromyogram (EMG) equipment, impedance-measurement equipment and other related applications (e.g., clinical applications and long-term health monitoring applications). Silver nanowires (AgNWs) can be embedded or otherwise positioned in polydimethylsiloxane (PDMS) to create a highly conductive stretchable and flexible network. PDMS can be used in biomedical applications due to its nontoxicity and high permeability to water and gas. Silver can be used in biomedical applications due to its antibacterial properties.

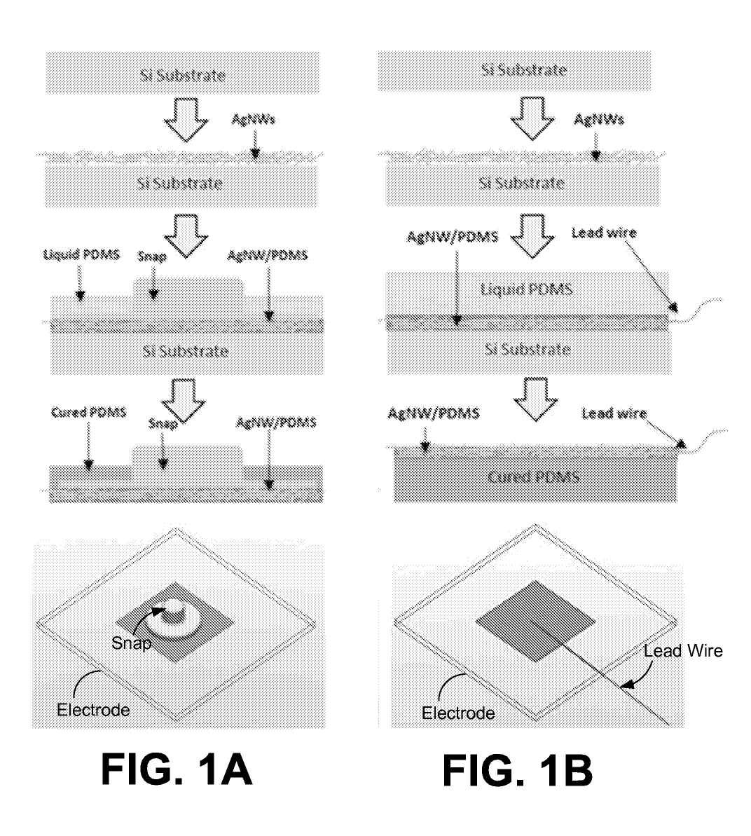

To fabricate a dry electrode, AgNWs can be cast onto a silicon substrate or suitable substrates, such as plastic and glass. After the solvent completely evaporates, a network of AgNWs remains and liquid PDMS is poured over the nanowires. At this stage, either a conductive element, such as a lead wire or a steel snap, is pressed on top of the AgNW/PDMS mixture. The substrate is then placed in vacuum to remove air bubbles from the PDMS and then cures in an oven at 100° C. for 1 h. When the cured PDMS is peeled off the substrate, the AgNW network is visibly bonded to the PDMS and the lead wire or snap are securely connected to the AgNW/PDMS network. A schematic diagram of the fabrication process for an electrode with a lead wire and an electrode with a snap is shown in FIG. 1. Velcro straps can be added to the electrodes to allow for the electrodes to be worn on the wrist.

An example benefit to using AgNWs embedded in PDMS is their ability to maintain high conductivity even when stretched. The surface resistance of the electrode can be measured in-situ as the electrode is strained from 0-50%. The resistance of the electrode stabilizes after repeated stretching and releasing meaning that the electrode will give consistent readings under varying strain conditions.

An issue with surface bioelectrodes is the skin-electrode contact. The skin's electrical properties are highly variable which leads to issues in acquiring consistent ECG readings and other similar readings. At low frequencies, the impedance of the skin is determined by the stratum corneum, the outermost layer of skin. While Ag/AgCl electrodes have a gel to help moisten this layer and improve electrode-skin contact, dry electrodes eliminate the use of this gel. Therefore, dry electrodes need a low skin-electrode impedance to attain signals of comparable quality to the Ag/AgCl electrodes. In addition, low skin impedance can help reduce motion artifacts, which are discrepancies in the ECG signal caused by movement. The skin-electrode impedance can be measured by performing a frequency sweep from 40 Hz-100 kHz using an impedance analyzer.

Applying pressure on the electrode can affect the resulting impedance trend. Although the impedance decreases with increasing application pressure, the benefits gained from increasing the pressure past a certain point are minimal. A force sensor can be used to measure the application pressure of the electrode on the skin. Three different pressure applications can be tested, including light (0.11 psi), medium (0.27 psi), and hard (1.72 psi). A medium pressure can be used when applying the electrodes for ECG tests as it may be the most comfortable for the subject while still applying enough pressure to obtain good ECG signals.

When taking an ECG with an Ag/AgCl electrode, a series of steps to remove the upper layer of the stratum corneum are followed consisting of removing the dead cells by abrasion and applying an extra electrolytic gel to hydrate the skin before applying the pre-gelled Ag/AgCl electrode. A benefit of a dry electrode is to minimize skin preparation, so no skin preparation is required before applying the AgNW/PDMS electrode for ECG testing. ECG signals can be measured using an ECG amplifier while the subject is resting. These signals can be taken with the AgNW/PDMS dry electrodes in the Lead I position and with commercial electrodes also in the Lead I position.

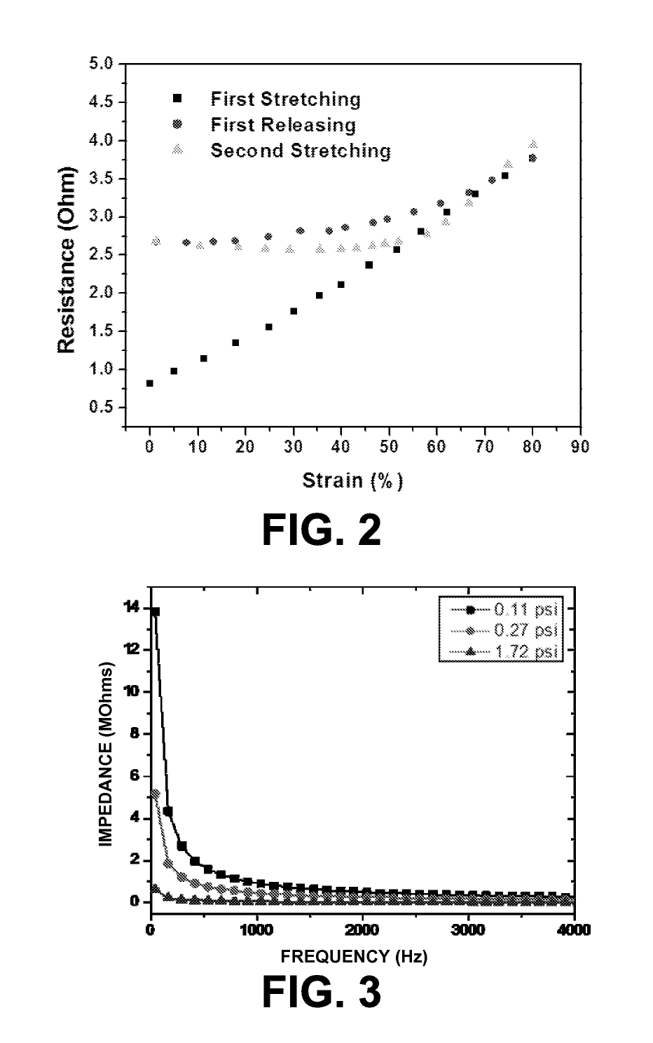

The resistance of the electrode, shown in FIG. 2, increased linearly with increasing strain and stabilized to approximately 8Ω after repeated stretching. The resistance value can be tailored by changing the density of the nanowires. This indicates that the AgNW/PDMS electrode can perform consistently under varying strain conditions, making it ideal for use in wearable or continuous health monitoring. The impedance-pressure trend is shown in FIG. 3. As the application pressure increases, the peak impedance decreases. This can be attributed to an increased contact surface area between the skin and electrode. The skin-electrode impedance values acquired are high, but do not affect the quality of the ECG signal.

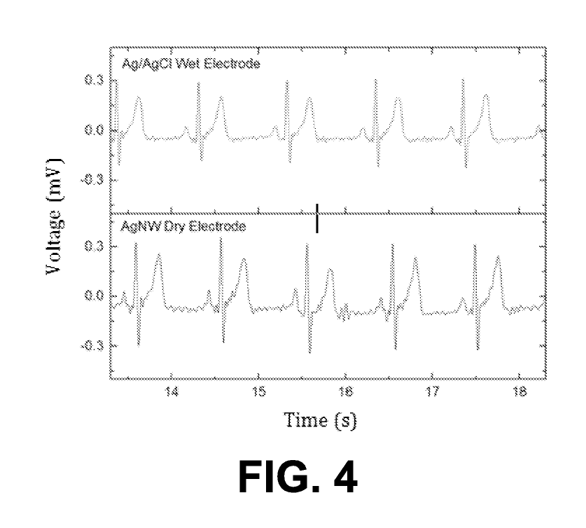

ECG signals can be acquired using the AgNW/PDMS electrodes and compared with signals acquired using conventional Ag/AgCl pre-gelled electrodes, as shown in FIG. 4. The signals were taken with the electrodes in the Lead I position, with the negative electrode placed on the right arm, the positive electrode placed on the left arm, and the ground electrode placed on the right leg. When acquiring ECG signals with the Ag/AgCl electrodes, the skin was cleaned and a small amount of electrolytic gel was added to the pre-gelled electrodes. However, no skin preparation or electrolytic gel was used when attaining ECG signals with the AgNW/PDMS electrodes. The P wave, QRS complex, and T wave show clearly in each of the ECG signals. These components are used for diagnostic purposes, so it is imperative that the waves can be viewed clearly. No critical differences between the AgNW/PDMS signal and the Ag/AgCl signal exist. When the extra electrolytic gel was not used with the pre-gelled electrodes, the signal acquired was of poorer quality than the AgNW/PDMS signal.

A silver nanowire based electrode for use in long-term ECG monitoring, EKG monitoring, or other suitable applications, can be fabricated. The AgNW/PDMS electrodes can be characterized by conductivity as a function of strain and by the electrode-skin impedance as a function of frequency and pressure. The conductivity of the AgNW/PDMS electrodes is retained throughout multiple 0-50% strains which show that the electrodes can maintain a high performance level in different strain/flex conditions. As expected, the initial impedance of the skin-electrode interface decreased with increasing application pressure. Although high skin-electrode impedance usually leads to a low-quality ECG signal, the AgNW/PDMS electrode yielded a high quality signal even though the skin-electrode impedance is high. The electrodes were then connected to an ECG amplifier to verify the ability to acquire high quality ECG signals. The ECG performance of each design of the AgNW/PDMS electrodes can be compared to conventional, pre-gelled Ag/AgCl electrodes and found to yield comparable results that can be used for diagnostic and monitoring purposes. The AgNW/PDMS electrode that was fabricated with a snap or contact was successfully connected to the ECG amplifier using conventional lead wire connections which will allow for use with conventional ECG machines already in existence. Further, the AgNW/PDMS electrode fabricated with a lead wire can be connected to lab-made ECG machines or to conventional ECG machines via the use of an alligator clip.

As disclosed herein, a dry electrode alternative to the widely-used, pre-gelled Ag/AgCl electrodes can be successfully fabricated and prove to be a viable choice for use in ECGs in both the clinical and continuous health monitoring settings. The AgNW/PDMS electrodes can be fabricated using an inexpensive method that has the potential to scale up to a large manufacturing assembly. The electrode design gives ECG signals of comparable quality to the Ag/AgCl electrodes without the skin preparation required of using the Ag/AgCl electrodes. The elimination of the electrolytic gel can allow for the AgNW/PDMS electrode to be worn for long periods of time without irritating the skin. The dry electrode design is compatible with current ECG equipment and will allow the electrodes to be easily integrated into existing biomedical devices at hospitals and clinics. The robust design of the AgNW/PDMS electrode can allow for reusability and will also allow it to be used in long-term monitoring.

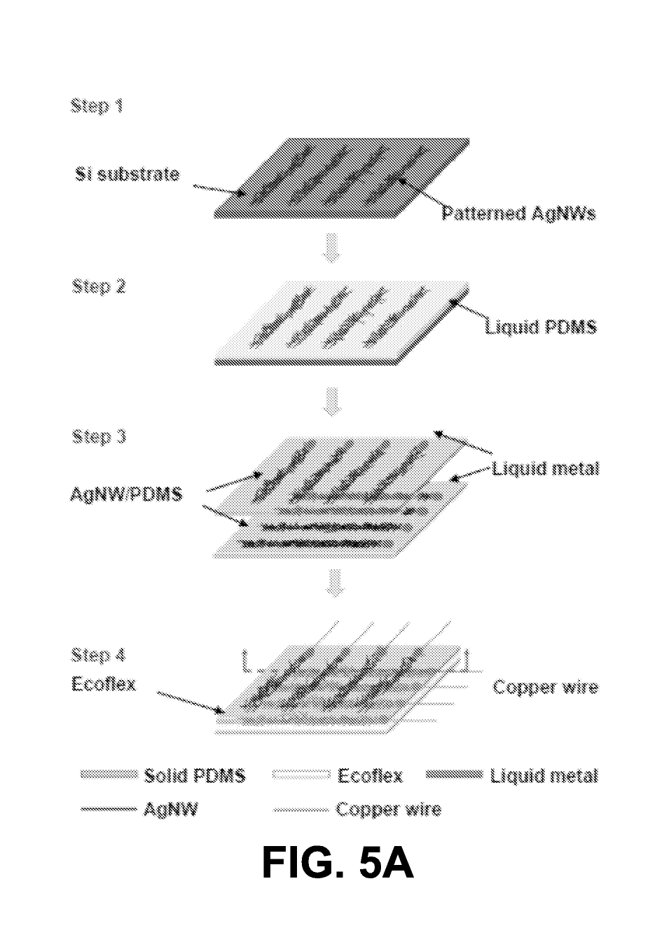

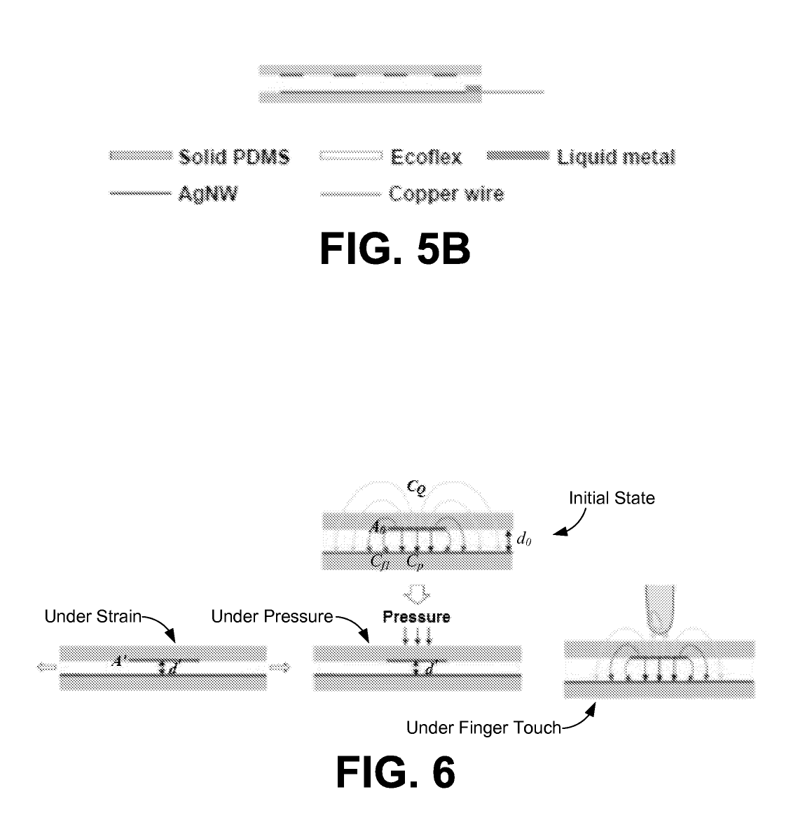

Initially, AgNW suspension in ethanol or other suitable solvents (e.g., water) can be drop-casted onto a pre-cleaned substrate and the metal NWs can then be dried to form a uniform and conductive coating of NWs. Such AgNW conductors can be patterned through a pre-patterned PDMS shadow mask, for example, with line width of ˜2 mm and spacing of ˜2 mm (Step 1 in FIG. 5A). Liquid PDMS (mixing the “base” and the “curing agent” with a weight ratio of 10:1) can then be casted onto the Silicon (Si) substrate that included the AgNW conductors on top, and cured at 65° C. for 12 hours. As a result, all the patterned AgNW conductors can be embedded just below the PDMS surface when it is peeled off the Silicon (Si) substrate (Step 2 in FIG. 5A). Eutectic gallium-indium (EGaIn, Aldrich, ≧99.99%) liquid metal can be applied to the two ends of the AgNW/PDMS strips to serve as conformal electrodes. After that, the AgNW/PDMS film can be positioned orthogonal to another identical AgNW/PDMS film face-to-face (Step 3 in FIG. 5A).

A soft dielectric layer (e.g., Ecoflex silicone elastomer) can be introduced as the dielectric layer of the capacitors. Liquid Ecoflex made by mixing part A and part B with the ratio of 1:1 can be applied between the two orthogonally positioned AgNW/PDMS films. At the same time, copper wires can be embedded inside the liquid metal and covered by Ecoflex liquid. Finally, the whole structure can be degassed in a vacuum oven followed by curing under ambient condition for approximately 4 hours (Step 4). This way, the Ecoflex layer can be sandwiched between the orthogonally patterned stretchable AgNW conductors to form the capacitive sensors. The AgNW, polydimethylsiloxane (PDMS) and dielectric layer as shown in FIG. 5B are about 5 μm, 0.2 mm and 0.5 mm in thickness, respectively, although other sizes can be implemented. In various embodiments, an individual capacitive sensor can be fabricated following the same process.

The capacitance was measured by an AD7152 capacitance-to-digital converters evaluation board. The principles for strain sensing, pressure sensing and touch sensing to be discussed later are schematically shown in FIG. 6.

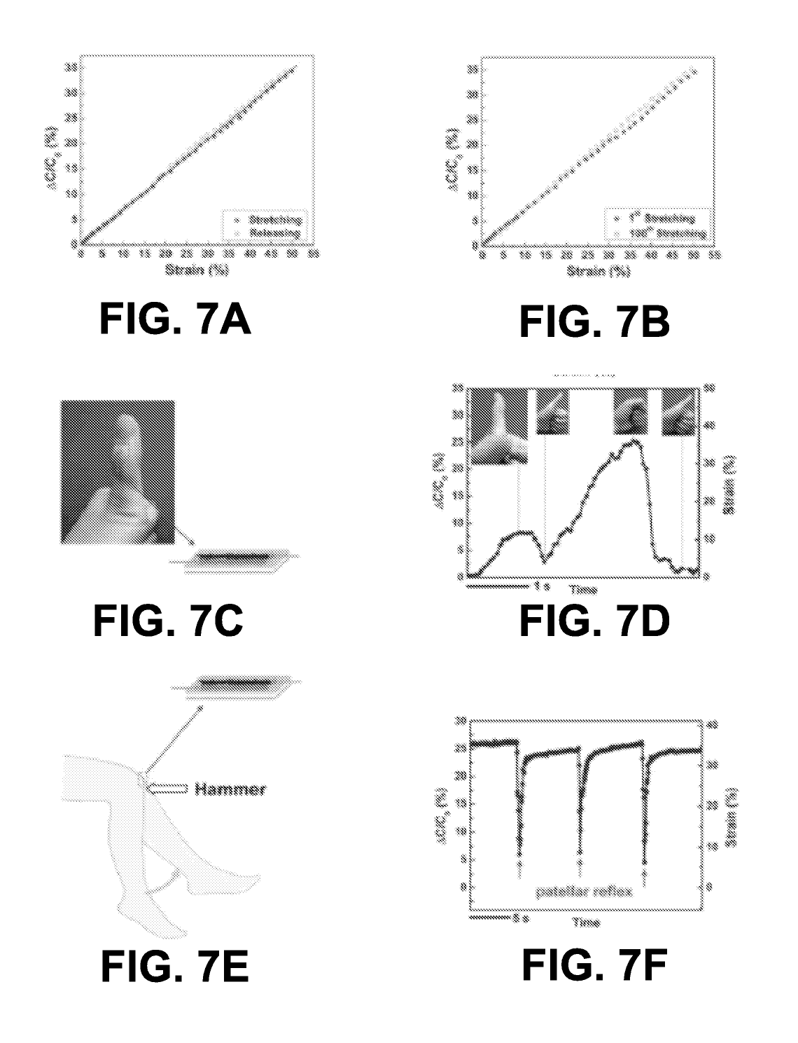

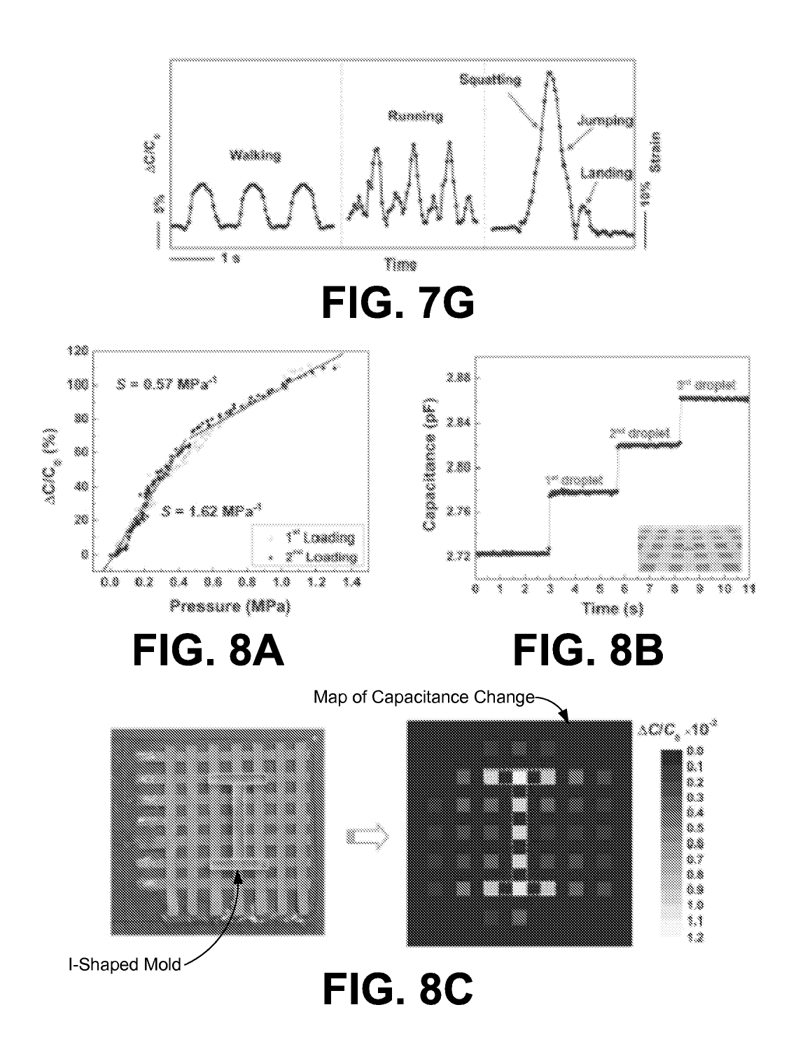

FIG. 7A shows the relative capacitance change ΔC/C₀ versus tensile strain during stretching and releasing. As the sensor is uniaxially stretched, the length (along the strain direction) of the electrode increases, while the width of the electrode and the separation between the two electrodes decrease, resulting in an increase in capacitance. The strain sensor exhibited good linearity and reversibility up to very large strain level (50%). In addition, the sensor exhibits excellent stability, as shown in FIG. 7B. The gauge factor (the relative change in capacitance divided by the mechanical strain) was found to be ˜0.7. Strain sensors based on resistive mechanism usually suffer from large hysteresis and nonlinearity under large strain. For the capacitive sensors described herein, the hysteresis was found to be negligible. In addition, the sensors can reliably detect the strain below 1%.

The strain range during human movement can be much larger than that of conventional strain gauges. In FIGS. 7C-7G, it is demonstrated through real-time strain measurements during large movements that the skin-mountable sensors as described herein can help monitor the body motions, which provides important information for feedback control in robotic systems, prosthetic devices, and other suitable uses. At the same time, the sensors can be beneficial for continuous health and wellness monitoring, for example, to help detect physiological conditions (such as knee-jerk) for diagnoses, monitor body motions during rehabilitation, and quantize the body movement to evaluate an athlete's performances.

In various embodiments, a matrix of capacitors, such as a 7×7 array of capacitors (“pixels”), can be fabricated following the process shown in FIG. 5A to form a pressure sensor that has spatial resolution. When a pressure is applied on the capacitor, the separation between the two AgNW layers decreases, leading to an increase in capacitance (as shown in FIG. 6B). The relative capacitance change ΔC/C₀ in FIG. 8A shows a bilinear dependence on the pressure. Compared with previously reported capacitive pressure sensors, the sensitivity of the sensors is higher than those with carbon nanotube electrodes (0.23 MPa-1 over the pressure range up to ˜1 MPa) and those with serpentine gold electrodes (0.48 MPa-1 over the pressure range up to 0.25 MPa), both using Ecoflex as dielectric layers. Pressure sensors composed of copper electrodes and an air gaps encapsulated by PDMS showed nonlinear response with a sensitivity of 3%/mN (4.8 MPa-1) over the range of 40 mN (250 kPa). The sensors with copper clad laminated composites on unstretchable polyimide substrate exhibited a sensitivity of 9.2 MPa-1 for the range of 40 kPa. Highly sensitive pressure sensors using microstructured PDMS dielectric layer and PET substrate can be achieved. The sensor showed similar bilinear response (0.55 kPa-1 for less than 2 kPa and 0.15 kPa-1 for 2-7 kPa), but on non-stretchable polyester substrate.

Fast response time is important in realizing real-time pressure monitoring. Small loadings can be applied by dispersing three 0.06 g water droplets, as shown in FIG. 8B. Here, the response time (rise time) can be defined as the time interval between 10% and 90% of the steady state values. Response time of the sensors, as described herein, was estimated to be around 40 ms, which is much shorter than those reported for other pressure sensors such as the flexible polymer foam based capacitive sensor (several seconds) and the one using an Gold (Au) film patterned on a PDMS membrane (200 ms). Very few pressure sensors can simultaneously achieve the large stretchability, fast response, high sensitivity, and good linearity. To demonstrate the function of measuring the spatial distribution of pressure, a 2.7 g mold with the shape of letter “I” was cut and placed onto the sensor. The resulting relative capacitance changes are plotted in FIG. 8C, where brightness corresponds to a higher capacitance change. As can be seen from FIG. 8C, the spatial distribution of the applied pressure is clearly identifiable.

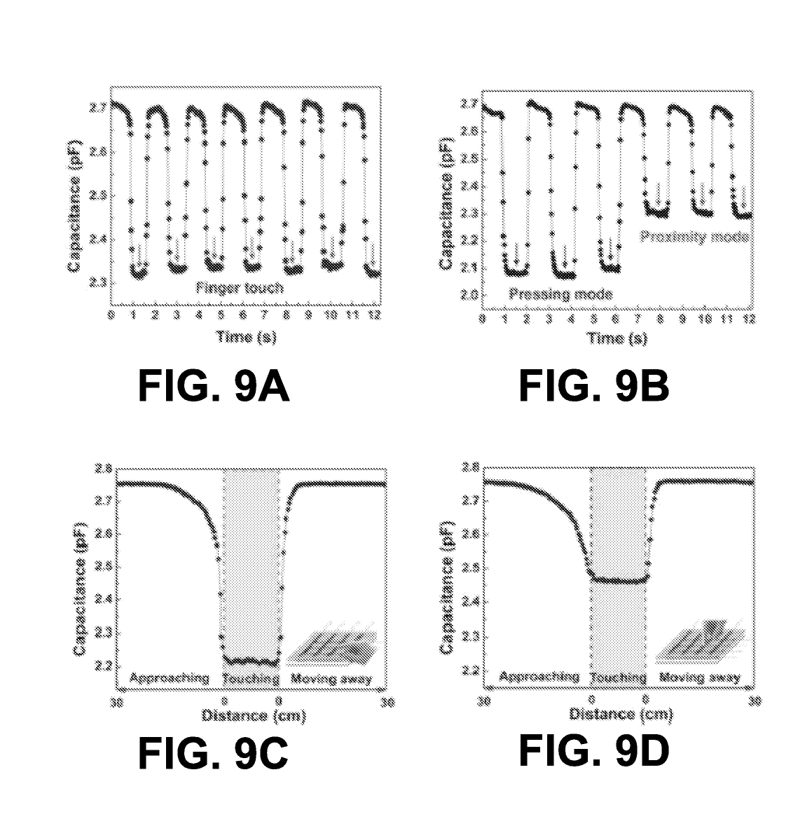

The capacitive sensors can also be used to detect finger touch and/or the touch of another grounded conducting medium, due to the partially grounded electric field by finger, as shown in FIG. 6C. The sensor can function well in both situations: (1) proximity mode (the finger is approaching, no force applied) and (2) pressing mode (force is applied). FIG. 9A shows the response of one pixel in the sensor array to finger touch (no force applied). As expected, the capacitance decreases upon finger touching. In order to probe the determining factors of the capacitance change, we approached the sensor from 30 cm away until touching with different finger areas. FIGS. 9C and 9D reveal that larger interaction area and shorter distance lead to a larger capacitance decrease because of the increased portion of the electric field intercepted by the finger.

In some touch sensing applications, forces from finger touch are inevitable. FIG. 9B presents the results for strong finger press (large force applied) and gentle finger touch (no force applied). The result indicates that the sensor can be reliably used as a touch sensor under either proximity mode or pressing mode (e.g., gentle or strong touches). For strong press, the interacting area between the finger and the pixel electrode is typically larger compared to gentle touch, which leads to a much larger decrease in capacitance. Flexible resistive and piezoelectric touch sensors have strechability limited either by the sensing layer or substrate material. Moreover, these sensors can only be used when the finger presses the sensors. In contrast, the capacitive touch sensors have a longer detecting range. This characteristic may be very useful in applications where contact between an electronic device and a human should be avoided or when contact between an electronic device and a human is undesirable.

By using stretchable materials for pressure sensors, the existence of tensile strain and normal pressure can be distinguished from the distribution of capacitance changes. Tensile straining affects all the pixels along the strain direction; in contrast, pressure only affects the pixels in the immediate vicinity of the load. The existence of the finger touch can also be identified and distinguished because only finger touch causes the decrease in capacitance. According to the specific needs, all the pixels can have the three functions or different pixels can be engineered to have different localized functions.

In various embodiments, the stability, sensitivity, linearity, detecting range, and/or response time can be further enhanced via optimization of geometry and materials. Further, the multifunctional sensors can be integrated with wearable devices (e.g., sensors, actuators, antennas, and power devices) and used as the conformal intelligent surfaces to interact with human and the environments in robotic systems, prosthetics, wearable health monitoring devices, or flexible touch pads.

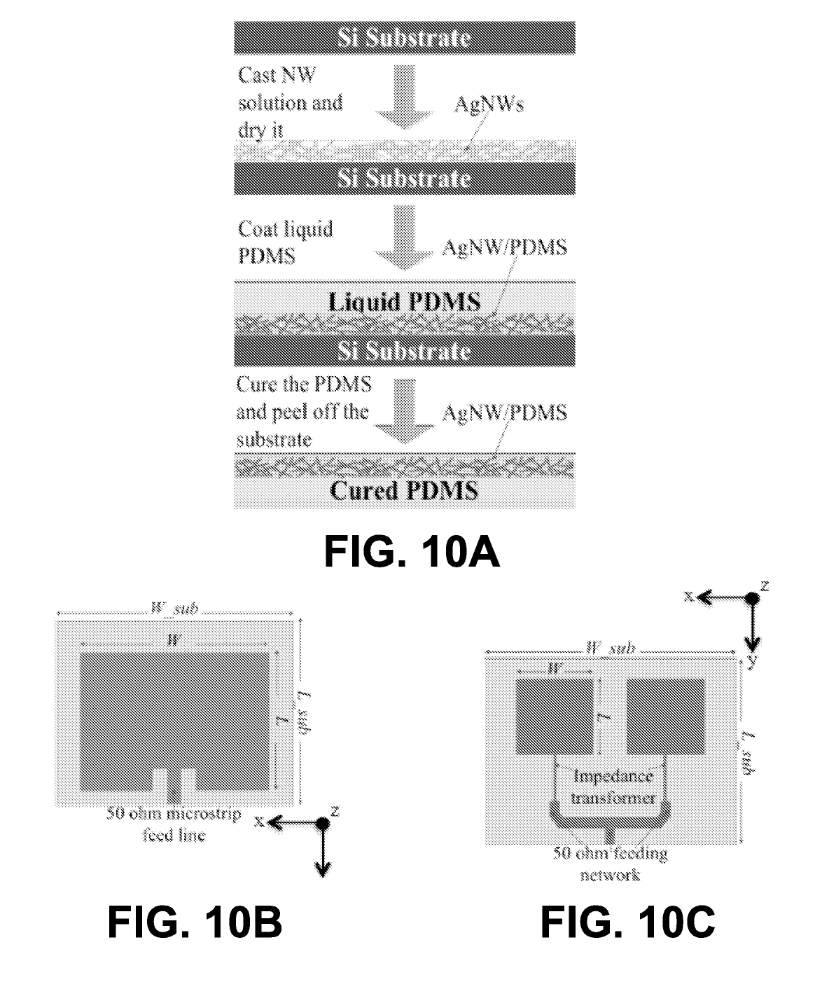

Moving on to FIG. 10A, shown is a fabrication procedure for the AgNW/PDMS patch antennas. Two types of patch antennas—the single patch (FIG. 10B) and 2-element array (FIG. 10C)—can be fabricated using the same or a similar process. The thickness of the AgNW/PDMS layer can be ˜0.5 mm and the separation between the radiating element and the ground plane can be ˜1 mm (±0.1 mm).

In various embodiments, the single patch antenna consists of a rectangular radiating patch, a ground plane, and a uniform layer of dielectric substrate between them. Dimensions of the patch can be designed based on a transmission-line model, which gives the width W and the length L as functions of the resonant frequency fᵣₑₛ, the relative permittivity of substrate material ∈ᵣ, and the thickness of substrate h. The substrate material PDMS has a reported relative permittivity ranging from ∈ᵣ=2.67 to 3.00 and loss tangent ranging from tan δ=0.01 to 0.05 over operating frequency range of 1.0 GHz to 5.0 GHz. Accordingly, the substrate material can be modeled with a relative permittivity of ∈ᵣ=2.80 and a loss tangent of tan δ=0.02 for a 3-GHz application.

Conductivity of the AgNW/PDMS stretchable conductor is ˜8,130 Scm-1 before stretching. Here, a constant conductivity of 8,130 Scm-1 can be used for the antenna considering the applied strains can be relatively small. To obtain the resonance frequency of 3 GHz, the rectangular patch was designed to be 36.0 mm×29.2 mm, backed with a 45.0 mm×40.0 mm ground plane, although other suitable sizes can be implemented. To match the input impedance with a 2.5 mm×8.0 mm 50Ω microstrip feed line, the inset feeding method was employed, which left a 3 mm external part and eliminated the need for an external matching network. Length and width of the cutout inset region were optimized in ANSYS HFSS to achieve lower return loss and less additional coupling between patch and feed line.

The 6-GHz 2-element array patch antenna can be designed similarly with the same material parameters except an increased loss tangent of tan δ=0.05. Two identical radiating elements with dimensions shrinking to 18.0 mm×14.3 mm can be arranged in parallel, and fed simultaneously by a feeding network. Since the doubled operating frequency renders the input impedance more sensitive to inaccuracy in dimensions, the matching strategy can be changed by introducing an impedance transformer at the edge of each radiating element to reduce possible fabrication error. Note that due to fabrication error, the obtained resonance frequencies for the patch and the 2-element array are 2.92 and 5.92 GHz, respectively.

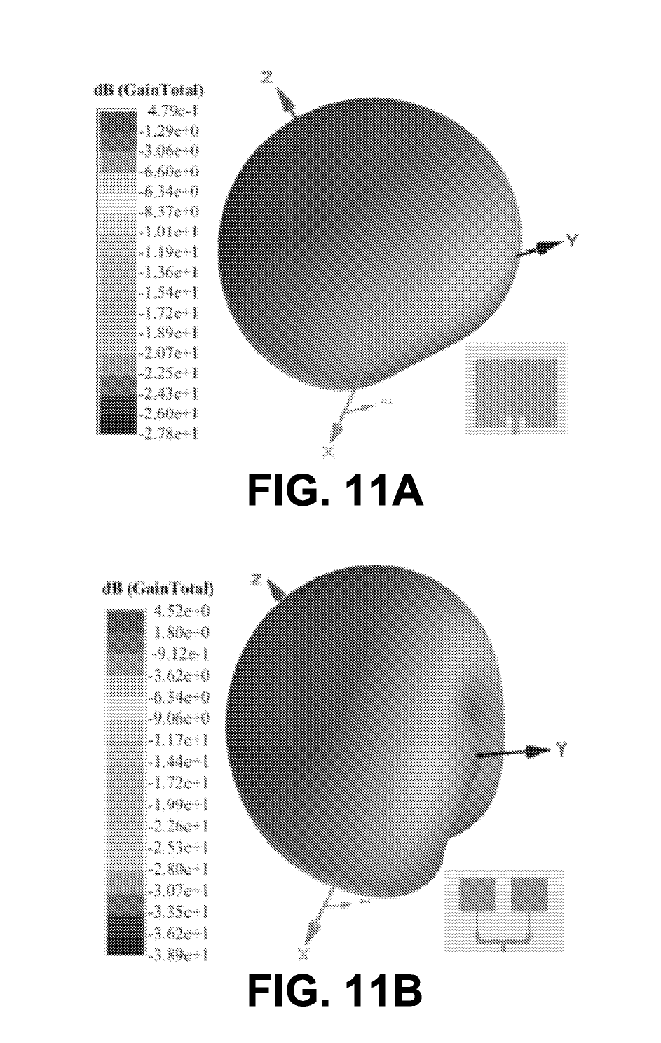

Simulated radiation patterns of the one and two element antennas were obtained by far-field calculation in ANSYS HFSS, as shown in FIGS. 11A-11B. Simulation results for the radiation properties of both antennas are summarized in Table 1 for comparison. The 2-element array, compared with the single element, increases the directivity by 4.5 dB and the fractional bandwidth by 2.5%, with higher radiation efficiency at the same time.

TABLE 1
Comparison of Simulated Radiation Properties for Microstrip
Patch Antenna and 2-Element Patch Array

| Radiation Properties | Monopole Patch | | 2-Element Patch Array | |
|---|---|---|---|---|
| Resonant Frequency | 2.92 | GHz | 5.92 | GHz |
| Peak Directivity | 4.16 | dBi | 8.14 | dBi |
| Peak Gain | 0.37 | dBi | 4.90 | dBi |
| Radiation Efficiency | 41.83% | | 48.83% | |
| Bandwidth | 88 | MHz (3.0%) | 330 | MHz (5.5%) |

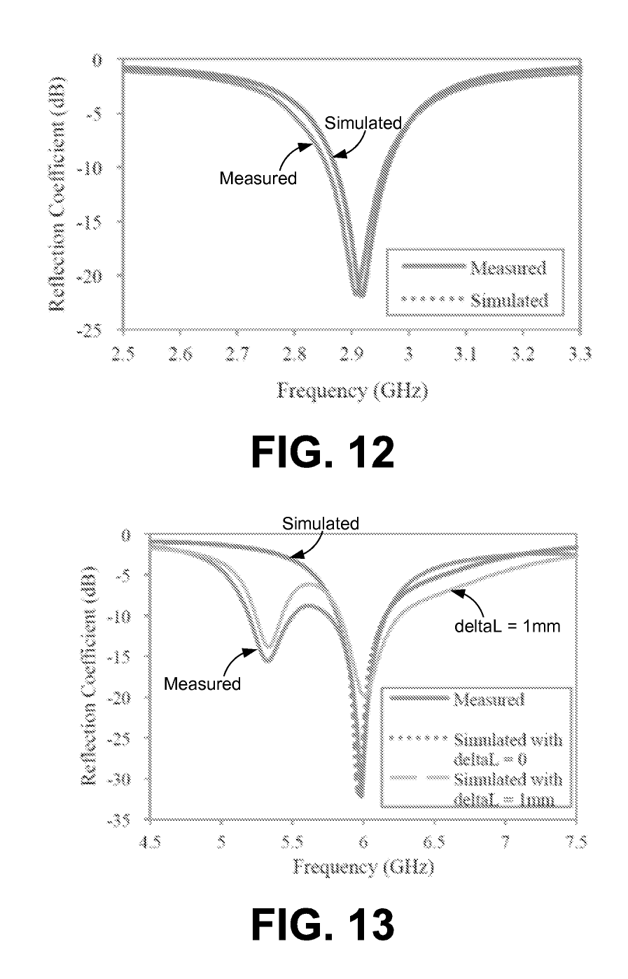

The patch antennas were characterized experimentally and compared to the simulated results. Measured and simulated frequency responses agreed very well, with the difference within the manufacturing imperfection and measurement uncertainty. FIG. 12 shows the measured spectrum response of the reflection coefficient over frequency range of 2 GHz to 4 GHz for the single patch antenna before stretching, with S11 below −20 dB at the center frequency of 2.92 GHz and above −1 dB far outside the operating region. The bandwidth, defined as the frequencies where S11<−10 dB, was 97.5 MHz.

The simulated reflection coefficient for the 2-element array was compared to the measured reflection coefficient from 4 GHz to 8 GHz. The array was initially designed with a single operating band at 6 GHz while the measured results showed an additional operating band at around 5.3 GHz. The mechanism of the unexpected resonance was studied by introducing small dimension deviations due to possible fabrication errors compared to the antenna model with ideal dimensions. As is shown in FIG. 13, when we assume that one of the radiating elements was fabricated larger in length than the other, the simulated frequency response would extend to the lower frequency band and form another band located at around 5.3 GHz for a 1 mm deviation in length, which was very close to what was observed experimentally.

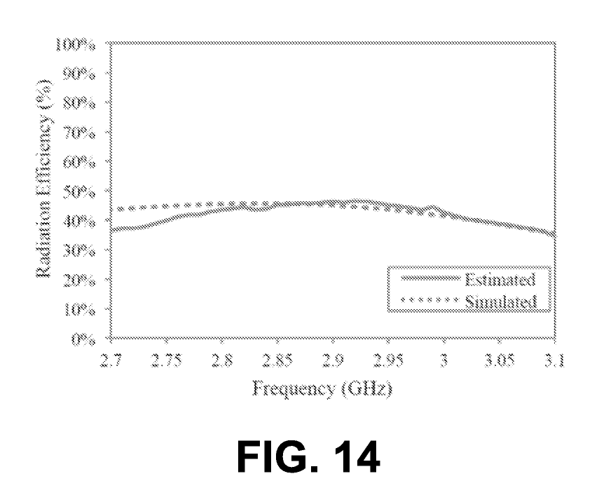

Far-field performance for the single patch antenna was tested in an anechoic chamber. The peak gain over frequency range of 2 GHz to 4 GHz was measured. Radiation efficiency was estimated using the measured gain and simulated directivity, which is compared to the simulated radiation efficiency in FIG. 14. To further study loss mechanisms of the AgNW/PDMS patch antenna with respect to radiation efficiency, antennas composed of four combinations of dielectric substrates and metal materials were modeled. Both AgNW/PDMS conductor with conductivity of 8130 Scm-1 and perfect electric conductor (PEC) with infinite conductivity for the metal components were considered. Also, both PDMS with loss tangent of tan δ=0.02 and lossless dielectric substrate was modeled. Table 2 summarized the simulation results of radiation efficiency for all the four combinations.

TABLE 2
Comparison of Simulated Radiation Efficiency of Antennas
in Different Dielectric and Metal Materials

| Substrate | Metal | Radiation Efficiency |
|---|---|---|
| Lossy PDMS | AgNW/PDMS | 41.53% |
| Lossy PDMS | PEC | 55.76% |
| Lossless Material | AgNW/PDMS | 67.20% |
| Lossless Material | PEC | 100% |

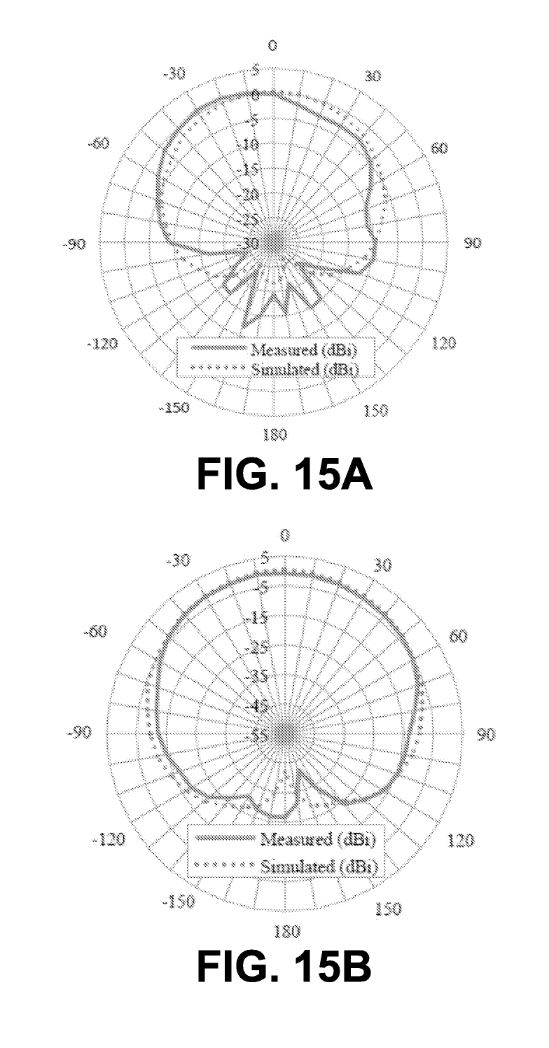

Compared to the ideal configuration, radiation efficiency was decreased from 100% to around 56% by the lossy substrate, and to around 67% by the AgNW/PDMS with finite conductivity. For completeness, the radiation pattern for the antenna in E-plane and H-plane is shown in FIG. 15. The stretchable antenna exhibits excellent radiation properties as well as good agreement with the simulated results.

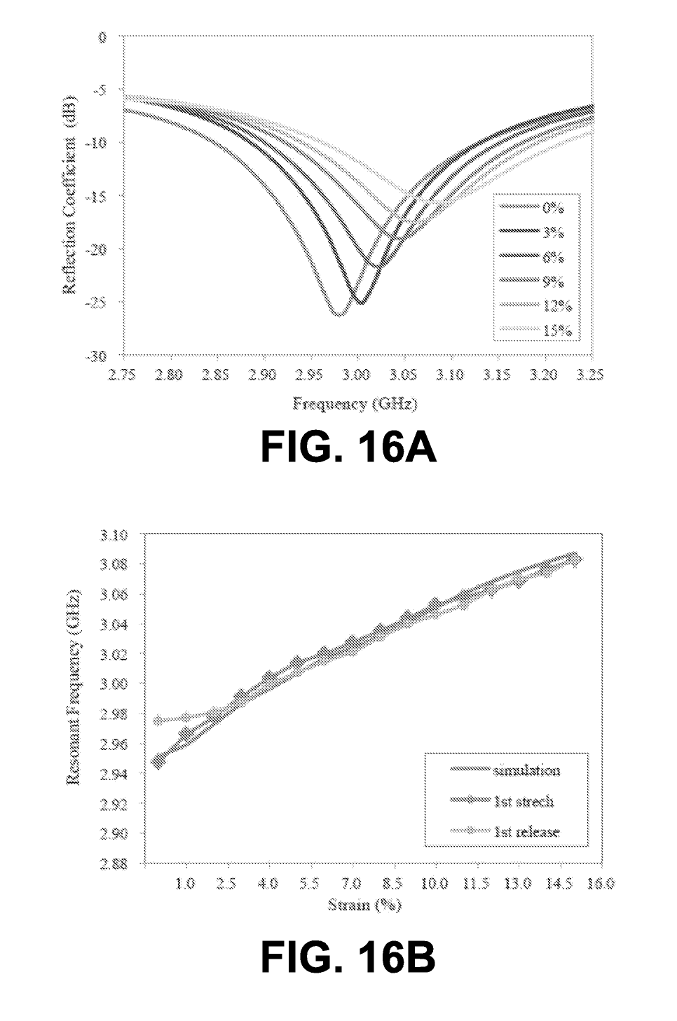

To test the mechanical tunability as a stretchable antenna, tensile strain ranging from 0% to 15% was applied to the AgNW/PDMS patch antenna in the width direction (perpendicular to the cable connection), while the reflection coefficient was collected by the network analyzer simultaneously. The antenna was tested on a custom-made mechanical testing stage, where all the components are made of insulators (e.g., ceramic, glass and Teflon). FIG. 16a shows the measured frequency response of the reflection coefficient under tensile strain from 0% to 5%. With the increasing strain, the spectrum response shifted to higher band, the center frequency increased almost linearly and the −10 dB bandwidth remained higher than 80 MHz, as listed in Table 3. The results suggest that performance of the stretchable antenna was not largely compromised during stretching.

TABLE 3
Measured Resonant Frequency and Bandwidth
of the AgNW/PDMS Microstrip Patch Antenna
Under Tensile Strains from 0% to 15%

| Strain (%) | 0 | 3 | 6 | 9 | 12 | 15 |
|---|---|---|---|---|---|---|
| Resonant | 2.947 | 2.991 | 3.020 | 3.044 | 3.063 | 3.083 |
| Frequency | | | | | | |
| (GHz) | | | | | | |
| Bandwidth | 239 | 244 | 253 | 254 | 243 | 258 |
| (MHz) | | | | | | |

The strain was then increased to 15% and slowly removed from the antenna. The center frequency was also measured during the releasing process. Upon complete release of the strain, the antenna returned to its original resonant frequency demonstrating excellent reversible deformability.

To analyze the frequency shift due to the applied strain, we accounted for the changing dimensions as functions of the strain. PDMS is a typical hyperelastic material where the total volume is constant during deformation. Therefore when the antenna is elongated in the width direction, the length and height shrink proportionally. The resonant frequency fᵣₑₛ is determined by the length of the radiating patch as:

$${f}_{res} = \frac{c}{2 L \sqrt{{∈}_{reff}}} , \qquad ( eq . 1 )$$

where c is the speed of light in vacuum, L is the length of microstrip patch antenna and ∈_{reff} is the effective relative permittivity of the microstrip, to account for the differing permittivities of the air and substrate material.

When a tensile strain of s is applied, the new dimensions of the antenna, patch width W, patch length L, and substrate thickness h as the function of s is:

$$\begin{matrix}W = {W}_{0} ( 1 + s ) , & ( eq . 2 ) \\ L = \frac{{L}_{0}}{\sqrt{1 + s}} , & ( eq . 3 ) \\ h = \frac{{h}_{0}}{\sqrt{1 + s}} , & ( eq . 4 )\end{matrix}$$

where W₀, L₀, and h₀ are the original dimensions before stretch, and for a small strain s<<1, the new resonant frequency is represented as:

$${f}_{res} = \frac{c \sqrt{1 + s}}{2 {L}_{0} \sqrt{{∈}_{reff}}} \approx \frac{c}{2 {L}_{0} \sqrt{{∈}_{reff}}} ( 1 + \frac{1}{2} s ) , \qquad ( eq . 4 )$$

which gives a linear relationship between the resonant frequency fᵣₑₛ to the applied strain s. The effective dielectric constant ∈_{reff} was updated for each strain level.

Results are compared to measurements during both stretching and releasing processes in FIG. 16b. The recorded center frequency as a function of tensile strain agrees well with what is predicted by modeling. The difference between simulated and measured resonant frequencies is within ±3 MHz at each point, which is relatively insignificant (within ±0.2%).

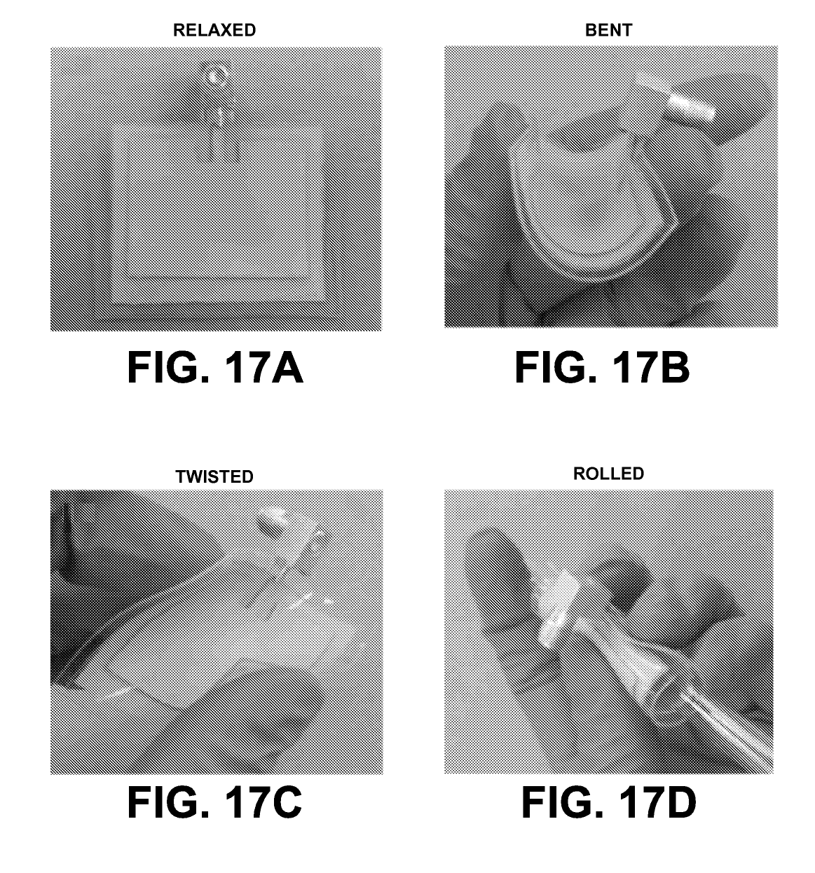

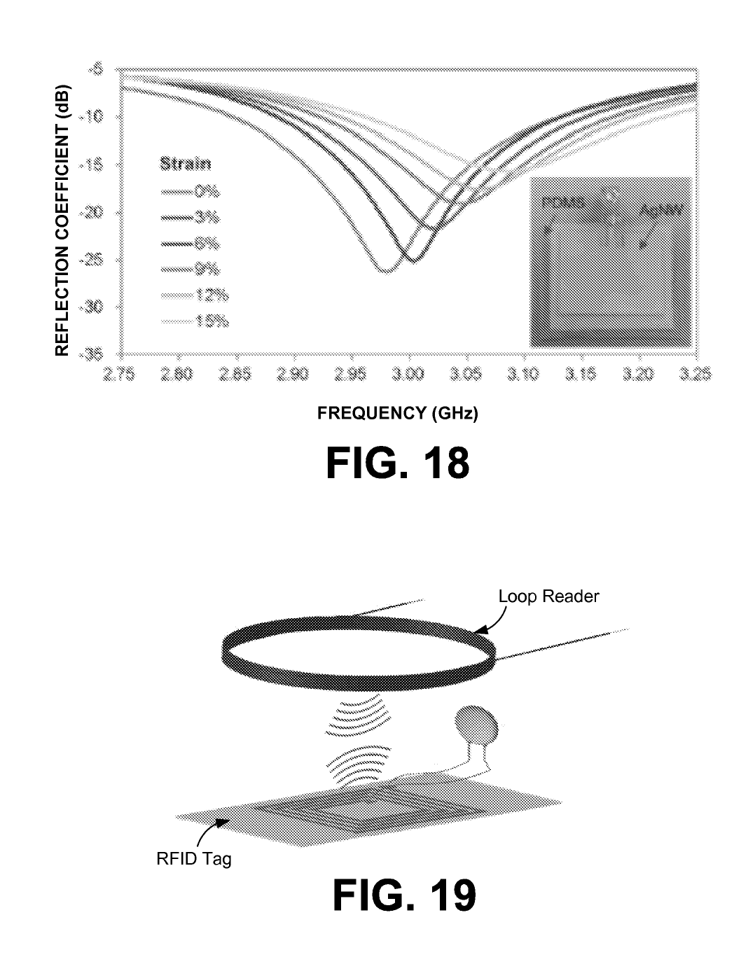

The stretchable antenna is thus well suited for wireless strain sensing applications. To further demonstrate the reversible deformability of the antenna, as described herein, the antennas can be subjected to other deformation modes including bending, twisting, and rolling, as shown in FIG. 17. For example, the antenna can be deformed by bending by 90° along the long axis, twisting along the long axis, rolling on both axes. After the deformed antenna is returned to its original state, it maintains almost the same spectral properties with difference of the resonant frequencies within ±1 MHz (<0.1%) before and after deformation in each case, which demonstrates that the AgNW/PDMS flexible antenna is reversibly deformable and robust.

It has been demonstrated that a class of microstrip patch antennas that are stretchable, mechanically tunable and reversibly deformable. A 3-GHz patch antenna and a 6-GHz 2-element patch array were fabricated. Radiating properties of the antennas were characterized under tensile strain, which agreed well with the simulation results. The antenna was mechanically tunable, enabling the resonant frequency to change as a function of the applied tensile strain. Thus it was well suited for applications like wireless strain sensing. The radiation efficiency was limited by losses in both the PDMS substrate and AgNW. The antennas were also demonstrated to maintain the same spectral properties after severe bending, twisting, and rolling. The material and fabrication technique reported here could be extended to achieve other types of stretchable antennas with more complex patterns and multi-layer structures.

In various embodiments, stretchable antennas can be fabricated as follows. AgNWs with average diameter of ˜90 nm and length in the range of 10-60 μm (or other suitable sizes) can be synthesized in a solution. For example, they can dispersed in ethanol with a concentration of 10 mg/mL. As shown in FIG. 10a, the antenna pattern can be cut out from a stencil mask on a pre-cleaned substrate such as silicon (Si) wafer. AgNWs solution was drop-casted into the mask on top of a hot plate (set at 55° C.) and then dried to form a conductive film of AgNWs with the desired antenna pattern. After peeling off the stencil mask, liquid PDMS was casted on top of the AgNW film, and heated at 100° C. for 1 hour to cure. When the cured PDMS layer was peeled off the substrate, the AgNW film was embedded into the PDMS forming a surface layer both conductive and stretchable. Fabrication of the ground plane layer can follow the same procedure, and then bond with the patch layer before the liquid PDMS cured.

In various embodiments, the stretchable antennas can be modeled and/or designed as follows. The width W and the length L are designed based on the transmission-line model.

$$\begin{matrix}W = \frac{c}{2 {f}_{res}} \sqrt{\frac{2}{{∈}_{r} + 1}} , & ( eq . 6 ) \\ L = \frac{c}{2 {f}_{res} \sqrt{{∈}_{reff}}} - 2 Δ L , & ( eq . 7 ) \\ {∈}_{reff} = \frac{{∈}_{r} + 1}{2} + \frac{{∈}_{r} - 1}{2} [ 1 + 12 \frac{h}{w} ] - \frac{1}{2} , & ( eq . 9 ) \\ Δ L = 0.412 h \frac{( {∈}_{reff} + 0.3 ) ( \frac{W}{h} + 0.264 )}{( {∈}_{reff} - 0.258 ) ( \frac{W}{h} + 0.8 )} , & ( eq . 10 )\end{matrix}$$

with ΔL as the “extended” length at each end because fringing fields at the patch edges make the length appear larger electrically than physically. For low frequencies (<10 GHz) the effective dielectric constant is essentially constant, referred to as the static values and given by eq. 8. Eq. 9 is a common approximate relation for the extension of length depending on the effective dielectric constant ∈_{reff} and the width-to-height ratio (W/h). Typically, ΔL<<L.

An antenna can be connected to a coaxial cable by a SMA connector. S-parameters can be collected using an Agilent E5071C Vector Network Analyzer to measure the resonant frequency and reflection coefficient. Radiation patterns were measured in the anechoic chamber at the NC State Remote Educational Antenna Lab (REAL). 2D pattern cuts were measured in the orthogonal E- and H-planes (YZ and XZ planes). Each cut was obtained by rotating the antenna under test (AUT) in 10 degree increments while recording the received signal with a broadband horn antenna (A.H. Systems) to produce the relative pattern plot. Absolute gain was calculated via gain comparison to a standard gain horn (A.H. Systems). The results given in the present disclosure represent the co-polar radiation patterns and gain.

Various types of strain sensors have been reported, which offer excellent performance in terms of strain range, sensitivity, linearity and stability. However, most of them require physical connection to external electronics and thus potentially limit their applications on moving objects or in sealed environment. It is therefore of interest to develop wireless strain sensors. In accordance with embodiments, a passive wireless strain sensor is provided that follows the principle of radio-frequency identification tag (RFID), based on our AgNW/PDMS stretchable conductors; see the schematic diagram in FIG. 19. The same patterning technology was first employed to fabricate a planar inductor on PDMS, as shown in FIG. 2A. By connecting the planar inductor with a dummy capacitor, an inductor-capacitor (LC) circuit was formed (FIG. 19). The LC circuit was remotely interrogated with a loop antenna via mutual inductance coupling between the planar inductor and loop antenna. The resonant frequency of the LC circuit was determined by measuring the real portion of the impedance spectrum across the terminals of the antennas by a precision impedance analyzer (Agilent 4294A). Tensile strains were then applied on the AgNW/PDMS based inductor, while the impedance spectra were recorded at the same time, from which the resonant frequency as a function of strain was extracted.

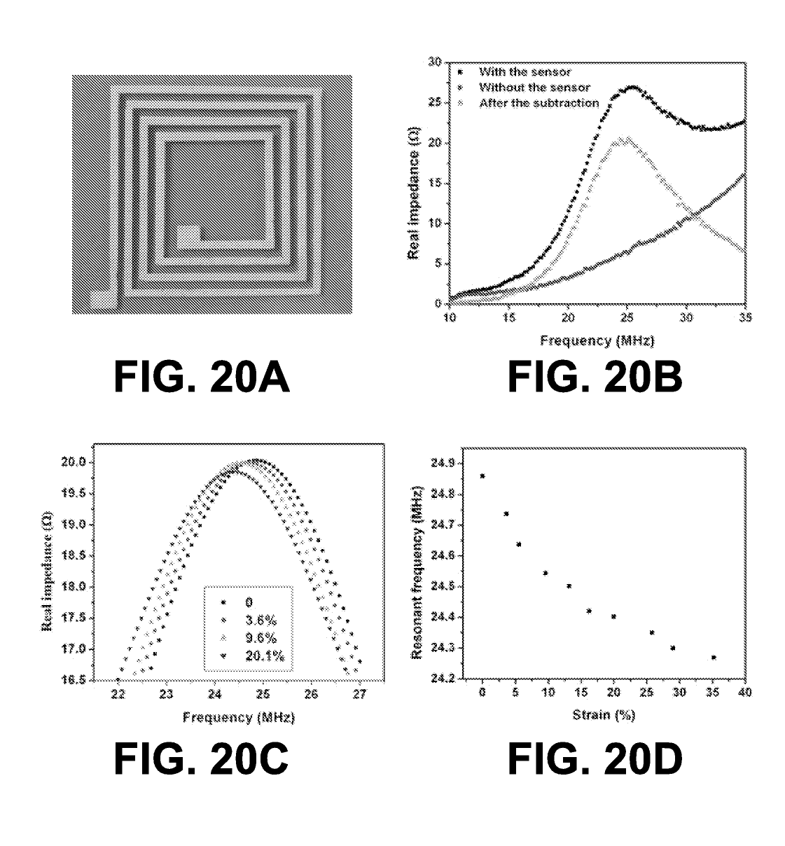

FIG. 20b shows the measured real impedance spectra in the frequency range of 5 to 40 MHz. The black and red curves represent the impedance spectra measured from the loop antenna with and without the LC sensor presented, respectively. By subtracting the red spectrum (i.e., background signal) from the black one, the intrinsic impedance of the loop antenna was removed from the signal and the net sensor response was obtained (the green curve in FIG. 20). When a strain was applied on the planar inductor, the resonant frequency changed in response to the change of the inductance, which was in turn due to geometric change of the planar inductor under strain. The inductance was found to increase with the tensile strain. Lorentzian fits of four representative real impedance spectra (only the portion around the peak from 22 MHz to 27.5 MHz) of the LC sensor under various strains are shown in FIG. 20c. The original data are provided in the Supplementary Information. It can be seen that the spectra shift gradually leftward with the applied tensile strain, which means that the resonance frequency of the LC sensor decrease with the tensile strain. The resonant frequency of the LC sensor was obtained from the fits and plotted in FIG. 20d as a function of the tensile strain. The resonant frequency of the LC strain sensor decreased monotonically with the strain up to 35.1%. The strain range was limited by the size of the loop antenna. A larger strain range could be achieved by using a larger loop antenna. Passive inductively coupled wireless strain sensors have been fabricated before; however, the strain range was limited. With the assistance of the AgNW/PDMS stretchable conductor, a large measurement range of 0-35.1% was achieved here, which is the largest strain range for any wireless strain sensor to the best of our knowledge.

While the embodiments have been described in connection with the preferred embodiments of the various figures, it is to be understood that other similar embodiments can be used or modifications and additions can be made to the described embodiment for performing the same function without deviating therefrom. Therefore, the disclosed embodiments should not be limited to any single embodiment, but rather should be construed in breadth and scope in accordance with the appended claims.

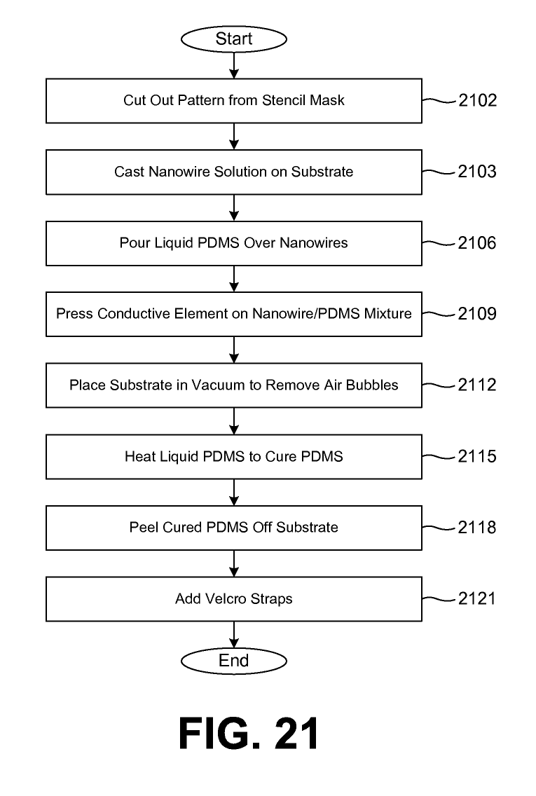

Referring next to FIG. 21, shown is a flowchart that provides one example of fabricating an electrode according to various embodiments. First, a pattern may be cut to create a stencil mask, such as a pre-cleaned substrate (2102). A plurality of nanowires, such as AgNWs or carbon nanotubes, are casted on the substrate (2103) cut out in 2102. In various embodiments, the substrate comprises silicon, plastic, glass, or any combination thereof. After the solvent has evaporated, a network of nanowires (e.g., AgNWs) remains and liquid PDMS is poured over the nanowires (2106) to create a mixture of nanowires and PDMS. Next, a conductive element (e.g., lead wire and/or a steel snap) is pressed on top of the nanowire/PDMS (e.g., AgNW/PDMS mixture)(2109). In various embodiments, the conductive element may be configured to operatively connect to photovoltaic equipment, a display device, and artificial skin. Similarly, in various embodiments, the conductive element may be configured to operatively connect to prosthetic equipment, a mechanical motion detector, pressure sensing equipment, a strain gauge, hydration sensing equipment, a biomorph actuator, or an actuator.

The substrate is then placed in a vacuum to remove air bubbles from the PDMS (2112). Then, the PDMS is heated to cure the PDMS (2115). In one example, the PDMS is cured in an oven at a suitable temperature for a suitable length of time. In various embodiments, the PDMS is cured in the oven at 100° C. for 1 h. Next, the cured PDMS is peeled off the substrate (2118), after which the AgNWs network is visibly bonded to the PDMS and the lead wire and/or the snap are securely connected to the AgNW/PDMS network. Finally, Velcro straps can be added to the electrodes to allow for the electrodes to be worn, for example, on the wrist (2121).

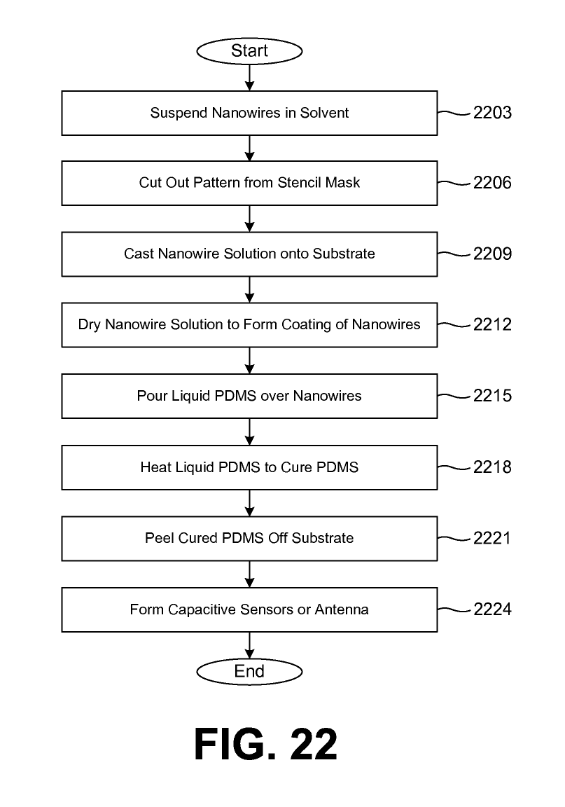

Referring next to FIG. 22, shown is a flowchart that provides one example of fabricating, for example, a stretchable antenna and/or a stretchable capacitive sensor according to various embodiments. In some embodiments, nanowires (e.g., AgNWs) are suspended in ethanol or other suitable solvents (e.g., water) (2203) to form a nanowire solution. However, in alternative embodiments, a pre-manufactured nanowire solution may be employed. Next, a suitable pattern, such as an antenna pattern or a capacitive sensor pattern, can be cut out from a stencil mask on a pre-cleaned substrate, such as a silicon (Si) wafer (2206). Next, the nanowire solution is drop-casted onto the stencil mask cut from the pre-cleaned substrate (2209). The solution drop-casted onto the substrate metal NWs are then dried to form a uniform and conductive coating of NWs (2212).

In various embodiments, the nanowire conductors can be patterned through a pre-patterned shadow mask (e.g., a PDMS shadow mask). For example, the nanowire conductors can be patterned with a line width of ˜2 mm and spacing of ˜2 mm. Liquid PDMS can then be casted onto the substrate that included the nanowire conductors on top (2215). The liquid PDMS is then cured at a suitable temperature for a suitable length of time (e.g., at 65° C. for 12 hours) (2218). The cured PDMS surface is then peeled off (2221). As a result, all the patterned nanowire conductors are embedded just below the PDMS surface when it is peeled off the substrate.

Finally, the antenna and/or capacitive sensor can be formed by applying two or more layers and/or arrangements of AgNWs/PDMS (2224). For example, the AgNW/PDMS film can be positioned orthogonal to another identical AgNW/PDMS film face-to-face. With respect to an antenna, the fabrication procedure for the AgNW/PDMS patch antennas of FIGS. 10A-C may be employed. For example, two types of patch antennas−the single patch (FIG. 10B) and 2-element array (FIG. 10C) can be fabricated using the flowchart of FIG. 22. In various embodiments, the thickness of the AgNW/PDMS layer can be ˜0.5 mm and the separation between the radiating element and the ground plane can be ˜1 mm (±0.1 mm).

With respect to a stretchable capacitive sensor, a soft dielectric layer (e.g., Ecoflex silicone elastomer) can be introduced as the dielectric layer of the capacitors. Liquid Ecoflex made by mixing part A and part B with the ratio of 1:1 can be applied between the two orthogonally positioned AgNW/PDMS films. At the same time, copper wires can be embedded inside the liquid metal and covered by a liquid, such as Ecoflex liquid. Finally, the structure can be degassed in a vacuum oven followed by curing under ambient condition for approximately 4 hours or other suitable length of time. As a result, the Ecoflex layer is positioned between the orthogonally patterned stretchable AgNW conductors to form the capacitive sensors.

Although the flowcharts of FIGS. 21 and 22 show a specific order of execution, it is understood that the order of execution may differ from that which is depicted, when feasible. For example, the order of execution of two or more blocks may be scrambled relative to the order shown. Also, two or more blocks shown in succession in FIGS. 21 and 22 may be performed concurrently or with partial concurrence. Further, in some embodiments, one or more of the blocks shown in FIGS. 21 and 22 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A sensor, comprising:
a dry electrode comprising a polymer material having a plurality of nanowires dispersed therein; and
a conductive element being attached to the electrode.

2. The sensor of claim 1, wherein the electrode is configured to measure skin to electrode impedance.

3. The sensor of claim 1, wherein a conductivity of the conductive element is retained during a strain of the sensor from 0% to 50%.

4. The sensor of claim 1, wherein the nanowires are silver nanowires.

5. The sensor of claim 1, wherein the nanowires are carbon nanotubes.

6. The sensor of claim 1, wherein the polymer material comprises a rubber substrate.

7. The sensor of claim 6, wherein the rubber substrate comprises polydimethylsiloxane (PDMS).

8. The sensor of claim 1, wherein the conductive element comprises a contact and a wire.

9. The sensor of claim 1, wherein the electrode and the conductive element are operatively connected to medical equipment.

10. The sensor of claim 9, wherein the medical equipment is selected from a group consisting of electrocardiogram (ECG) equipment, electrocardiography (EKG) equipment, electroencephalogram (EEG) equipment, electromyogram (EMG) equipment, and impedance-measurement equipment.

11. The sensor of claim 1, wherein the electrode and the conductive element are communicatively coupled to one of photovoltaic equipment, a display device, and artificial skin.

12. The sensor of claim 1, wherein the electrode and the conductive element are communicatively coupled to at least one of prosthetic equipment, a mechanical motion detector, pressure sensing equipment, a strain gauge, hydration sensing equipment, a biomorph actuator, or an actuator.

13. A method for creating a dry sensor, comprising:
casting a plurality of nanowires onto a substrate;
pouring a liquid form of polydimethylsiloxane (PDMS) over the plurality of nanowires to create a mixture of the plurality of nanowires and the PDMS; and
pressing a conductive element on the mixture, the conductive element being configured to communicatively couple to medical equipment.

14. The method of claim 13, further comprising placing the substrate in a vacuum to remove air bubbles from the PDMS.

15. The method of claim 13, further comprising curing the PDMS in an oven at 100° C. for 1 hour.

16. The method of claim 13, further comprising peeling a cured portion of the PDMS off the substrate.

17. The method of claim 13, wherein the plurality of nanowires are silver nanowires or carbon nanotubes.

18. The method of claim 13, wherein the medical equipment is selected from a group consisting of: electrocardiography (EKG) equipment, electroencephalogram (EEG) equipment, electromyogram (EMG) equipment, and impedance-measurement equipment.

19. The method of claim 13, wherein the conductive element is operatively connected to one of photovoltaic equipment, a display device, and artificial skin.

20. The method of claim 13, wherein the conductive element is operatively connected to one of prosthetic equipment, a mechanical motion detector, pressure sensing equipment, a strain gauge, hydration sensing equipment, a biomorph actuator, and an actuator.
